(19) United States
(12) Patent Application Publication
(10) Pub. No.: US 2025/0305913 A1
(43) Pub. Date: Oct. 2, 2025

(54) METHOD AND APPARATUS FOR ANALYSING STEAM QUALITY

(71) Applicant: STERIS EUROPE, INC., Tuusula (FI)

(72) Inventor: Juha Mattila, Tuusula (FI)

(21) Appl. No.: 18/863,256

(22) PCT Filed: May 25, 2023
(86) PCT No.: PCT/FI2023/050294

§ 371 Date: Nov. 5, 2024

(30) Foreign Application Priority Data
May 25, 2022 (FI) 20225458

(51) Int. Cl. G01N 1/22; G01N 1/40; G01N 27/08; G01N 33/18

(52) U.S. Cl. CPC G01N 1/2247; G01N 1/4022; G01N 27/08; G01N 33/18; G01N 2001/4033

(57) ABSTRACT

The invention provides an apparatus and method for analysing the purity of a steam flow in a steam line (1). A sample of the steam flow from the steam line (1) is directed into a sampling pocket (4) and condensed into water in the sampling pocket. The purity of the condensed sample is determined (5) and the sample is returned back to the steam line (1).

### FIELD

The present application relates to a method and apparatus for analysing purity of a steam flow in steam line. More precisely, the present application relates to an analysis of the purity of steam flow in a steam line.

### BACKGROUND

Pure steam is generally used in the pharmaceutical and medical industry. Pure steam can be used in for example sterilization, decontamination, sanitization and pasteurization. In these applications it is critical that the pure steam does not contain any pyrogens, particles or chemicals. Therefore, it is important to analyse the purity of the pure steam.

To analyse the purity of the pure steam a sample has to be drawn from the pure steam line. The sample has to be condensed to water. Typically, the condensing is done by a heat exchanger using cooling water. The condensed sample is then analysed with for example, a conductivity sensor and then drained into the sewer. Some examples of analysing the purity of the pure steam are presented in German patent application DE102017125246A1, Chinese utility models CN206132450U and CN210051737U.

The problem with the current solutions is that they can consume a large amount of water, even up to 120 000 liters per year, as they use water for cooling and the sample is drained after the analysis, the response time between the purity measurement and detecting the contaminated steam can be long and the condensing the pure steam into analyzable water can be energy consuming.

The embodiments of the present invention are intended to overcome at least some of the above discussed disadvantages and restrictions of the prior art.

### SUMMARY OF THE INVENTION

The invention is defined by the features of the independent claims. Some specific embodiments are defined in the dependent claims.

According to a first aspect of the present invention, there is provided a method for analysing the purity of a steam flow in a steam line. The method comprises directing a part of the steam flow from the steam line and into a sampling pocket, wherein said directed part constitutes a sample of steam, condensing the sample of steam into water in the sampling pocket, determining the purity of the condensed sample, and returning the sample back to the steam line.

According to a second aspect of the present invention, there is provided an apparatus for analysis of purity of a steam flow in a steam line. The apparatus comprises a sampling pocket integrally connected to and in fluid connection with the steam line, means for condensing a sample in said sampling pocket, and a conductivity sensor configured to determine the conductivity of a condensed steam sample in the sampling pocket, wherein said sampling pocket is configured to receive a part of a steam flow from the steam line and to return said part back to the steam line.

According to a third aspect of the present invention, there is provided a method for analysing the conductivity of a steam flow in a pure steam line or a clean steam line, comprising: generating steam in a pure steam generator or a clean steam generator; feeding the steam to a pure steam line or a clean steam line to provide a steam flow in said steam line; directing a part of the steam flow from the steam line into a sampling pocket, wherein said directed part constitutes a sample of steam; condensing the sample of steam into water in the sampling pocket; determining the conductivity of the condensed sample; and returning the condensed sample back to the steam line.

Various embodiments of the first aspect or the second aspect or the third aspect may comprise one or more features from the following bulleted list:
The purity of the condensed sample is determined by determining the conductivity of the sample with a conductivity sensor.
The steam line is a steam line configured to transport pure steam, which pure steam has a conductivity of less than 3.1 μS/cm measured at 95° C., when condensed.
The steam line is a steam line configured to transport pure steam, such as steam having a conductivity less than 5 μS/cm, such as less than 1.5 μS/cm.
The sample of steam is a flow of 0.5-3.5 l/h, such as 1-3 1/h, for example 0.5-1.0 l/h.
The sampling pocket is integrally connected to and in fluid connection with the steam line.
The method comprises directing said part of the steam flow out of the steam line at a first location.
After said condensing and determining steps, the method comprises returning the sample to the steam line at a second location.
The second location is downstream in the steam line with regard to the first location.
The directed part of the steam flow forms a loop.
The temperature of the condensed sample is lowered to the range 95-100° C. in the sampling pocket.
The condensing step comprises cooling the sample.
The cooling and condensing of the sample is done by an electrically powered cooling chiller.
An electrically powered chiller connected to the sampling pocket is used for condensing and cooling of the sample.
The electrically powered cooling chiller is operated at a power of 400-600 W in said condensing step.
The design of the sampling pocket creates a pressure drop or subjects the sample flow entering the sampling pocket to a pressure drop, which condenses the sample flow into water or at least facilitates the condensing of the sample flow into water.
The returning of the sample back to the steam line comprises vaporizing the sample into steam, for example by using an ejector.
The sampling time of the purity analysis is less than 5 minutes, for example less than 4 minutes, such as less than 3 minutes.
The method is carried out continuously during transport of steam in the steam line.
The steam comprises or consists of pure steam.
The steam comprises or consists of clean steam.
The steam comprises or consists of saturated steam.
The steam comprises or consists of pressurized saturated steam.
The pure steam or saturated steam has a pressure in the range 1 to 8 bar, such as 2 to 5 bar.
The pure steam or saturated steam has a temperature of at least 100° C.
The pure steam or saturated steam has a temperature in the range 100 to 180° C.
The condensing of the sample is carried out by a device arranged to condense the sample, typically by cooling the sample without any cooling water.
Said cooling is carried out by air-mediated cooling.
Said cooling is carried out by a refrigerant-based cooler, such as a closed loop refrigerant based cooler.
Said cooling is carried out by a Peltier element.
Said cooling is carried out by a thermoelectric cooler, such as a TEG.
The returning step comprises returning the sample to a returning location in the steam line.
Said returning location is downstream and separate from the location in the steam line at which said directing into a sampling pocket takes place.
An input of the sampling pocket is connected to the steam line at a first location, and an output of the sampling pocket is connected to the steam line at a second location, such as a returning location.
The second location is preferably downstream in the steam line with regard to the first location.
The sampling pocket is made of stainless steel.
The apparatus further comprises or the output of the sampling pocket comprises or consists of an ejector for enabling vaporizing the sample into steam and returning it back to the steam line or which is configured to vaporize the condensed sample into steam upon returning the sample back to the steam line.
The conductivity sensor is configured to determine conductivities at least in the range 1-5 μS/cm, such as 0.8-1.2 μS/cm.
The apparatus is configured to have sampling time of the purity analysis of less than 5 minutes, for example less than 4 minutes, such as less than 3 minutes.
The means for condensing the sample is an electrically powered cooling chiller.
The electrically powered cooling chiller is configured to be operated at a power of 400-600 W.
The means for condensing the sample is a refrigerant-based cooler.
The sampling pocket is configured to provide a pressure drop for condensing the pure steam sample.
The steam line is a pure steam line or a clean steam line.
Said generating step comprises generating saturated steam from water.
Said generating step comprises generating saturated steam by a pure steam generator.
Said generating step comprises generating saturated steam by a clean steam generator.
The steam flowing in the pure steam line is saturated steam.
The steam flowing in the pure steam line has a pressure in the range 1 to 8 bar.
The steam flowing in the pure steam line has a temperature in the range 100 to 180° C.
The method is carried out continuously during operation of the pure steam generator, such as during generation of steam by the generator and generally also during idle running of the generator.

According to a fourth aspect of the present invention, there is provided use of the method according to the first aspect or the apparatus according to the second aspect or the method according to the third aspect in sterilization, decontamination, sanitization and/or pasteurization.

The current invention may provide several advantages.

The present process may save water as it may avoid using cooling water and the sample may be returned back to the pure steam line after analysis. Also, the sampling time of the sampling measurement is significantly reduced. The sampling time of the sampling measurement can be reduced to about 50% of the conventional sample time.

An advantage is that the process may only consume a small amount of electric power to operate, such as only about 400-600 W.

### BRIEF DESCRIPTION OF THE DRAWINGS

FIG. 1 illustrates an operating principle of the apparatus and method in accordance with at least some embodiments of the present invention.

### EMBODIMENTS

### Definitions

In the present context, the term “pure steam” comprises steam which is substantially free of pyrogens, particles and chemicals. Pure steam has a conductivity of less than 3.1 μS/cm measured at 95° C., when condensed.

Generally, pure steam comprises or consists of saturated steam.

Typically, pure steam or saturated steam has a pressure in the range 1 to 8 bar, such as 2 to 5 bar.

Typically, pure steam or saturated steam has a temperature in the range 100 to 180° C., such as 100 to 151° C., such as 121.1 to 151° C. In an embodiment, the temperature of saturated steam is below 138° C.

In an embodiment, pure steam fulfils the requirements of the standard EN 285:2015+A1:2021, particularly with regard to dryness, super-heat property, and non-condensable gas content of the steam.

In some embodiments, pure steam is generated by a pure steam generator. The term “pure steam generator” refers to a steam generator arranged to generate pure steam. The generator receives water as an input, typically water that has been purified by reverse osmosis or deionization. In the generator, the water is vaporized in a column to produce pure steam.

In preferred embodiments, the quality and purity of the generated steam is analysed by determining the conductivity of the generated steam, preferably in a continuous manner.

The conductivity is a good indicator for purity of the steam and can be determined for example with an online conductivity meter without any manual sampling and analysis in an external laboratory.

Typically, the term “pure steam” refers to steam that is free from pyrogens, particles and chemical contaminants.

Typically, the term “clean steam” refers to steam that is free from chemical contaminants, such as boiler chemicals or other chemical additives.

The term “free from pyrogens” typically refers to “free from microbiological contaminants and free from microorganisms or their parts”.

Sometimes pure steam also can be referred to as “clean steam”.

The present invention may be applicable for pure steam analysis as well as clean steam analysis.

In the present context, the term “sampling time” is the time that it takes for the whole sample amount in the sampling pocket to be replaced by a new sample amount. For example, if a sampling pocket size is 0.8 litres and the sample flow rate is 6 litres per hour, the sample time is 8 minutes.

We have found a new way to obtain a sample from a steam line for analysis.

Particularly, we have found a method for analysing the purity of the steam flow in a steam line, which method preferably does not consume water and only uses a small amount of energy.

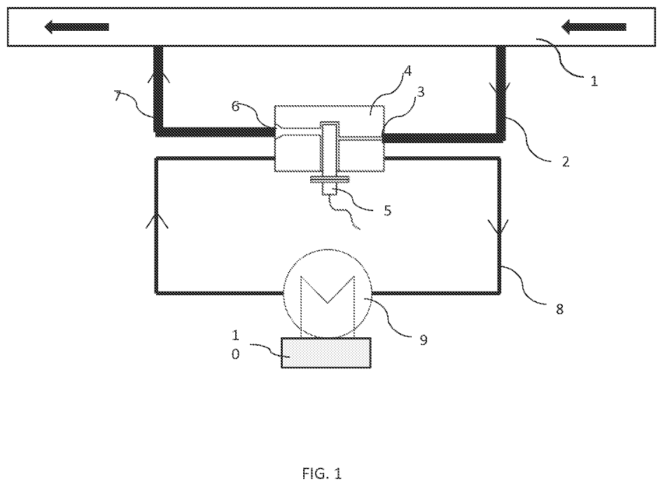

FIG. 1 illustrates an operating principle of the apparatus and method in accordance with at least some embodiments of the present invention. The flow chart describes a pure steam line 1 having a bypass line 2 for redirecting a sample of steam into a sampling pocket 4. The sample of steam is then condensed into water and the conductivity of the sample is analysed.

The steam line 1 carries pure steam from the pure steam generator. A sample of the steam is directed to the steam sample line 2, which carries the sample to the sampling pocket 4 though the orifice 3. The orifice 3 creates a pressure drop to the sample flow, which causes the steam sample to cool and condense to water. The cooling is enhanced by cooling the sampling pocket 4 with a chiller cooling media circulation 8. The cooling media 8 is chilled by an electric chiller 9 having an air-cooled condenser 10.

The condensed sample passes a conductivity sensor 5 that measures the conductivity of the steam sample. The conductivity measurement defines the quality of the sample and therefore the quality of the steam in the steam line 1. Once the steam sample has passed the conductivity sensor 5, it can be vaporized by an ejector 6. The vaporized sample is returned back to the steam pipeline 1 through a sample return line 7.

Embodiments of the invention are described next. Each of the features described below may be used in connection with the embodiment described in FIG. 1 or in other embodiments.

In an embodiment, the steam is generated by means of a point-of-use steam generator or by means of a central steam generator.

The conductivity of the water that is fed to the steam generator is typically less than 5 μS/cm.

In one embodiment, the temperature of the condensed sample is at least 80° C., preferably 95-100° C. The conductivity sensor used can be any suitable conductivity sensor or some other specific conductivity sensor.

The sample analysis may be carried out as a continuous process and it is done in-line. The sample flow is 0.5-3.5 l/h, such as 1-3 l/h, for example 0.5-1 l/h. There is no need to transport the condensed steam sample to a laboratory for further analysis.

In an embodiment, the sample flow is at least 0.5 l/h, such as 0.5-8 l/h, such as 0.5-6 l/h, such as 0.5-3.5 l/h, such as 1-3 l/h, for example 0.5-1 l/h.

The conductivity of the sample should be between 1 and 5 μS/cm, preferably between 0.8 and 1.2 μS/cm. If the conductivity is greater than this, the steam is not pure and it cannot be used.

Preferably, the conductivity of the sample should be between 0.8 and 5 μS/cm.

Particularly, for clean steam the conductivity of the sample should be between 1 and 5 μS/cm.

Typically, for pharmaceutical applications, such as for pure steam, the conductivity is preferably between 0.8 and 1.2 μS/cm.

By having a continuous in-line sample analysis with a short condensing line the sampling time is significantly reduced. The sampling measurement sampling time of the process is less than 5 minutes, for example less than 4 minutes, such as less than 3 minutes. Therefore, the safety of the process is increased and the amount of wasted steam and energy is decreased.

After the analysis the sample is directed back to the steam line using an ejector. The small sample amount will not affect the pure steam in the pure steam line.

According to an embodiment of the current invention there is provided a method for analysing the purity of a steam flow in a steam line, comprising:
directing a part of the steam flow from the steam line and into a sampling pocket, wherein said directed part constitutes a sample of steam;
condensing the sample of steam into water in the sampling pocket;
determining the purity of the condensed sample; and
returning the sample back to the steam line.

According to an embodiment the purity of the condensed sample is determined by determining the conductivity of the sample with a conductivity sensor. The conductivity can be determined by a specific conductivity sensor or other similar design type of combination of a conductivity sensor and a conductivity analyzer that only requires a minimal sample amount for conductivity measurement. The conductivity sensor is embedded into the sampling pocket.

According to an embodiment, the steam line is a steam line configured to transport pure steam, such as steam having a conductivity less than 5 μS/cm, such as less than 1.5 μS/cm.

According to an embodiment, the sample of steam is a flow of 0.5-3.5 l/h, such as 1-3 l/h, for example 0.5-1.0 l/h.

According to an embodiment, the sampling pocket is integrally connected to and in fluid connection with the steam line. Between the sampling pocket and the steam line there may be a steam sample line to connect the sampling pocket orifice to the steam line and the sampling pocket ejector to the steam line.

According to an embodiment, the method further comprises directing said part of the steam flow out of the steam line at a first location, and after said condensing and determining steps, returning the sample to the steam line at a second location, which is preferably downstream in the steam line with regard to the first location.

According to an embodiment, the directed part of the steam flow forms a loop. The loop comprises the steam sample line, the sample return line and the sampling pocket.

In some embodiments, the returning step comprises returning the sample to a different location (a returning location) in the steam line, preferably by utilizing an ejector. Typically, said returning location is downstream and separate from the location in the steam line at which said directing into a sampling pocket takes place.

According to an embodiment, the temperature of the condensed sample is lowered to the range 95-100° C., such as 95 to 99° C., in the sampling pocket. The temperature is such that the sample is condensed from steam to water.

According to an embodiment, the condensing step comprises cooling the sample.

According to an embodiment, the condensing of the sample is carried out by a device arranged to condense the sample by cooling the sample, preferably without any cooling water, such as without any closed-loop circulating cooling water or without any through-flowing cooling water.

According to an embodiment, said cooling is carried out by air-mediated cooling or by a refrigerant-based cooler, such as a closed loop refrigerant based cooler, or by a Peltier element or by a thermoelectric cooler, such as a TEG.

According to an embodiment, the cooling and condensing of the sample is done by an electrically powered cooling chiller. The electrically powered chiller is connected to the sampling pocket.

In an embodiment, the electrically powered cooling chiller is operated at a power to condense the sampled steam in said condensing and/or cooling step.

According to an embodiment, the electrically powered cooling chiller is operated at a power of 400-600 W in said condensing and/or cooling step. The chiller leads cooling refrigerant or alternatively internal loop re-circulating cooling water amount through the sampling pocket and this way condensing the required sample amount from steam to water for conductivity measurement.

In an embodiment, the device arranged to condense the sample, such as the cooling chiller, is electrically operated and applies a closed-loop refrigerant, without any water consumption or cooling water.

According to an embodiment, the design of the sampling pocket creates a pressure drop or subjects the sample flow entering the sampling pocket to a pressure drop, which condenses the steam into water or at least facilitates the condensing of the sample flow into water. The design of the sampling pocket creates a pressure drop by applying a sudden flow channel and inflow orifice dimension reduction. In other words, the diameter of the orifice is smaller than that of the sample line, which created a pressure drop in the sample flow. By utilizing the design of the sampling pocket, the need for excess cooling of the sample is minimized and therefore the need for energy is decreased.

According to an embodiment, the returning of the analysed sample back the steam line comprises vaporizing sample into steam, for example by using an ejector. As the sample amount is so small, the vaporized water does not substantially affect the temperature or purity of the steam in the steam line. The steam flow in the steam line creates a suction so that the sample flow is drawn towards the flow in the steam line. The sample can be vaporized before the sample flow connects to the flow in the steam line or once the sample flow is connected to the flow in the steam line by the pressure and temperature of the surrounding steam.

According to an embodiment, the sampling time of the purity analysis is less than 5 minutes, for example less than 4 minutes, such as less than 3 minutes. As defined above, this is the time it takes for the whole sample amount in the sampling pocket to be replaced by a new sample amount.

According to an embodiment, the method is carried out continuously during transport of steam in the steam line. In other words, steam flows continuously to the steam sample line and further through the sampling pocket to the conductivity sensor for conductivity determination.

Preferably, the present method is an automated method.

Typically, the present method is carried out continuously during operation of the pure steam generator, such as during generation of steam by the generator and generally also during idle running of the generator.

According to an embodiment, there is provided an apparatus for analysis of purity of a steam flow in a steam line, the apparatus comprising:
a sampling pocket integrally connected to and in fluid connection with the steam line;
means for condensing a sample in said sampling pocket; and
a conductivity sensor configured to determine the conductivity of a condensed steam sample in the sampling pocket,
wherein said sampling pocket is configured to receive a part of a steam flow from the steam line and to return said part back to the steam line.

According to an embodiment, an input of the sampling pocket is connected to the steam line at a first location and an output of the sampling pocket is connected to the steam line at a second location, which second location is preferably downstream in the steam line with regard to the first location.

According to an embodiment, the sampling pocket is made of stainless steel.

According to an embodiment, the apparatus further comprises or the output of the sampling pocket comprises or consists of an ejector for enabling vaporizing the sample into steam and returning it back to the steam line or which is configured to vaporize the condensed sample into steam upon returning the sample back to the steam line. As the sample amount is so small, the vaporized sample does not substantially affect the temperature or purity of the steam in the steam line. The steam flow in the steam line creates a suction so that the sample flow is drawn towards the flow in the steam line. The sample can be vaporized before the sample flow connects to the flow in the pure steam line or once the sample flow is connected to the flow in the pure steam line by the pressure and temperature of the surrounding steam.

According to an embodiment, the conductivity sensor is configured, such as calibrated, to determine conductivities of at least 0.1 μS/cm.

According to an embodiment, the conductivity sensor is configured to determine conductivities at least in the range 0.1-20 μS/cm, for example 1-5 μS/cm, preferably 0.8-1.2 μS/cm.

The conductivity measurement value is provided to the control system for further process use. If the conductivity measurement is larger than a reference value, the sample flow has impurities. Therefore, the steam flow has to be terminated and pipelines cleaned.

According to an embodiment, the apparatus is configured to have a sampling time of the purity analysis of less than 5 minutes, for example less than 4 minutes, such as less than 3 minutes. As defined above the sampling time is the time that it takes for the whole sample amount in the sampling pocket to be replaced by a new sample amount.

According to an embodiment, wherein the means for condensing the sample is an electrically powered cooling chiller.

According to an embodiment, the electrically powered cooling chiller is configured to be operated at a power of 400-600 W. The chiller leads cooling refrigerant or alternatively internal loop re-circulating cooling water amount through the sampling pocket and this way condensing the required sample amount from steam to water for conductivity measurement.

According to an embodiment, the sampling pocket is configured to provide a pressure drop for condensing the pure steam sample. The design of the sampling pocket creates a pressure drop by applying a sudden flow channel and inflow orifice dimension reduction. In other words, the diameter of the orifice is smaller than that of the sample line, which created a pressure drop in the sample flow. By utilizing the design of the sampling pocket, the need for excess cooling of the sample is minimized and therefore the need for energy is decreased.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e. a singular form, throughout this document does not exclude a plurality.

### INDUSTRIAL APPLICABILITY

The present invention is industrially applicable for example in applications where pure steam is used, for example in the pharmaceutical industry.

### REFERENCE SIGNS LIST

1 Steam pipeline
2 Steam sample line
3 Orifice
4 Sampling pocket
5 Conductivity sensor
6 Ejector
7 Sample return line
8 Chiller cooling media circulation
9 Electric chiller
10 Air-cooled condenser

### CITATION LIST

### Patent Literature

DE102017125246A1
CN206132450U
CN210051737U

1. A method for analysing the purity of a steam flow in a steam line, comprising:
directing a part of the steam flow from the steam line and into a sampling pocket, wherein said directed part constitutes a sample of steam;
condensing the sample of steam into water in the sampling pocket;
determining the purity of the condensed sample; and
returning the sample back to the steam line, wherein the steam line is a steam line configured to transport pure steam, such as steam having a conductivity less than 5 μS/cm, such as less than 1.5 μS/cm.

2. The method according to claim 1, wherein the purity of the condensed sample is determined by determining the conductivity of the sample with a conductivity sensor.

3. (canceled)

4. The method according to claim 1, wherein the sample of steam is a flow of 0.5-3.5 l/h, such as 1-3 l/h, for example 0.5-1.0 l/h.

5.-7. (canceled)

8. The method according to claim 1, wherein the temperature of the condensed sample is lowered to the range 95-100° C. in the sampling pocket.

9. (canceled)

10. The method according to claim 91, wherein the condensing of the sample is done by an electrically powered cooling chiller.

11. (canceled)

12. The method according to claim 10, wherein the electrically powered cooling chiller is operated at a power of 400-600 W in said condensing step.

13.-14. (canceled)

15. The method according to claim 1, wherein the sampling time of the purity analysis is less than 5 minutes.

16. (canceled)

17. The method according to claim 1, wherein the steam comprises saturated steam.

18. The method according to claim 17, wherein the saturated steam has a pressure in the range 1 to 8 bar.

19. The method according to claim 1 wherein the saturated steam has a temperature in the range 100 to 180° C.

20. The method according to claim 1, wherein the condensing of the sample is carried out by a device arranged to condense the sample by cooling the sample without any cooling water.

21.-22. (canceled)

23. An apparatus for analysis of purity of a steam flow in a steam line, the apparatus comprising:
a sampling pocket made of stainless steel and integrally connected to and in fluid connection with the steam line;
means for condensing a sample in said sampling pocket; and
a conductivity sensor configured to determine conductivities at least in the range 1-5 μS/cm, such as 0.8-1.2 μS/cm and configured to determine the conductivity of a condensed steam sample in the sampling pocket,
wherein said sampling pocket is configured to receive a part of a steam flow from the steam line and to return said part back to the steam line.

24. The apparatus according to claim 23, wherein
an input of the sampling pocket is connected to the steam line at a first location, and
an output of the sampling pocket is connected to the steam line at a second location, which second location is preferably downstream in the steam line with regard to the first location.

25. (canceled)

26. The apparatus according to claim 23, wherein the apparatus further comprises or the output of the sampling pocket comprises or consists of an ejector for enabling vaporizing the sample into steam and returning it back to the steam line or which is configured to vaporize the condensed sample into steam upon returning the sample back to the steam line.

27. (canceled)

28. The apparatus according to claim 23, wherein the apparatus is configured to have sampling time of the purity analysis of less than 5 minutes, for example less than 4 minutes, such as less than 3 minutes.

29. The apparatus according to claim 23, wherein the means for condensing the sample is an electrically powered cooling chiller.

30. The apparatus according to claim 23, wherein the electrically powered cooling chiller is configured to be operated at a power of 400-600 W.

31. The apparatus according to claim 23, wherein the means for condensing the sample is a refrigerant-based cooler.

32. The apparatus according to claim 23, wherein the sampling pocket is configured to provide a pressure drop for condensing the pure steam sample.

33. (canceled)

34. A method for analysing the conductivity of a steam flow in a pure steam line, comprising:
generating saturated steam from water fed into a pure steam generator;
feeding the steam to a pure steam line at a pressure in the range 1 to 8 bar and a temperature in the range 100 to 180° C. to provide a steam flow in said pure steam line;
directing a part of the steam flow from the pure steam line into a sampling pocket, wherein said directed part constitutes a sample of steam;
condensing the sample of steam into water in the sampling pocket;
determining the conductivity of the condensed sample; and
returning the condensed sample back to the pure steam line.

35.-38. (canceled)
